(19) United States
(12) Patent Application Publication
(10) Pub. No.: US 2004/0086873 A1
(43) Pub. Date: May 6, 2004

(54) System and method of generating and storing correlated hyperquantified tissue structure and biomolecular expression datasets

(76) Inventors: Peter C. Johnson, Wexford, PA (US); Andres Kriete, Pittsburgh, PA (US); Keith S. Boyce, Wexford, PA (US); Ronald A. Stone, Pittsburgh, PA (US); Andrew Lesniak, Pittsburgh, PA (US)

Correspondence Address: REED SMITH LLP, 2500 ONE LIBERTY PLACE, 1650 MARKET STREET, PHILADELPHIA, PA, 19103, US

(21) Appl. No.: 10/286,478

(22) Filed: Oct. 31, 2002

(51) Int. Cl. C12Q 1/68; G06F 19/00; G01N 33/48; G01N 33/50

(52) U.S. Cl. 435/6; 702/20

(57) ABSTRACT

We disclose a method for the correlation of structural feature information derived from digital images of a diagnostic set of histopathological tissue specimens from a group of subjects with shared characteristics and molecular activity (which may be either gene or protein expression) information derived from related specimens of the same set to provide for the objective characterization and analysis of the variability of tissue structural features in relation to such molecular activity (the product of such correlation referred to herein as the “correlated tissue information”). Such correlated tissue information allows for comparison and combination with correlated tissue information obtained through studies taking place at different times, with different protocols for the collection, preservation, histological staining and molecular activity assays of the tissue. The present invention specifically relates to a system and method to obtain and store correlated tissue information by combining hyperquantified feature expression data with gene and/or protein expression data. Such a system also allows for the relation of such correlated tissue information to certain clinical diagnoses or conditions through the inclusion or exclusion parameters describing the shared characteristics of the set of subjects providing the tissue specimens for analysis.

The present application claims priority to: U.S. patent application Ser. No. 09/338,909, entitled, Methods For Profiling And Classifying Tissue Using A Database That Includes Indices Representative Of A Tissue Population, filed Jun. 23, 1999; U.S. patent application Ser. No. 09/338,908, entitled, Online That Includes Indices Representative Of A Tissue Population Database, filed Jun. 23, 1999; U.S. patent application Ser. No. 10/106,582, entitled, Quantification And Differentiation Of Tissue Based Upon Quantitative Image Analysis, filed Mar. 25, 2002; and to U.S. patent application Ser. No. 10/158,486, entitled, Robust Stain Detection And Quantification For Histological Specimens Based On A Physical Model For Stain Absorption, filed May 29, 2002; all of which are incorporated by reference herein for all purposes.

### FIELD OF THE INVENTION

The present invention generally relates the correlation of the structure with the biomolecular activity of a tissue.

### BACKGROUND OF THE INVENTION

A common belief is that biomolecular expression is responsible for cellular responses and the resulting structural composition of tissue. However, it is also commonly believed that the underlying gene networks, protein-protein interactions and regulatory wirings and the resulting biochemical and biophysical behavior of a tissue reflected in its composition and organization are mostly of a non-linear nature, and asynchronous in time.

Analysis of gene expression data and state of the art cluster analysis alone may indicate functional classes of genes causing up-stream regulatory effects. By discounting the linearity of or a chronology to the resulting structure of the tissue, possibly to avoid the arduous task of collecting comparable data quantifying the features of the relevant structural elements of that structure, this gene-centric approach, by accepting genetic causality sorted by clinical diagnosis but not refined and confirmed by correlated tissue feature expression, has limited predictive power. In particular, lack of comparable quantification of structural variability, and the lack of re-measurement over time, prevents the interpretation of dynamical processes, such as the reaction of tissues to toxic compounds or the development of particular diseases, on whatever timescale they occur.

Microarray analysis technologies to generate highly quantified biomolecular expression data are well established. However, processes to acquire data describing the features of the structural elements of a tissue of comparable quantity and in a complementary form are not available. As a result, the uncertainty, imprecision, time, and subjectivity involved in making manual measurements of features characterizing those structural elements precludes meaningful correlation of biomolecular expression with tissue structure to identify and isolate the regulatory effect of particular genes or proteins.

A detailed deciphering of the essential components comprising these regulatory networks, their dynamics, and the emergent biological properties of the formed tissue can only be obtained when quantitative information of tissue feature expression is available in a numerical form comparable in breadth to related biomolecular data. Hyperquantitative analysis provides such quantitative data at the tissue structural level, to objectively profile, in a preferred embodiment, the net effects of the influence of toxic or test compounds or disease progression on the underlying gene and protein expressions and biomolecular network dynamics and to discriminate among those expressions to assign a higher probability of association between specific genes or proteins and discrete structural changes.

Prior approaches to the understanding of the influence of genes on tissue feature expression (normal or diseased) have attempted to correlate genetic molecular expression data with an overall interpretation of the tissue structure in the form of a pathological diagnosis. This approach is flawed because: diagnosis is a consensus descriptor that allows multiple similar—but not identical—tissue appearances to be grouped together; specimen-specific genetic expression data is likely to correlate more directly with the structure of the particular feature expression of that specimen than to that of the overall group of specimens carrying the same diagnosis. In addition, microarray-based genetic expression analysis has inherent variability, which is compounded when multiple specimens with the same diagnosis are analyzed and compared to pooled normals. This prior approach makes it difficult to identify the true genetic expression pattern that causes the disease state by obscuring the subtle but potentially significant structural variability between tissues, even within a well-defined specimen set. There is also no way to use this data to determine the sub-diagnostic structural influences of gene expression patterns because the most refined correlate possible is the overall diagnosis itself.

The visually derived microscopic diagnosis of tissue as assigned by a pathologist can be considered to be the summing up of all visible cues within the tissue image. Since these visual cues are all biologically relevant elements, the recreation of this diagnosis as a highly annotated, quantitative set of data describing the discrete tissue elements and their relative mathematical values correlated to biomolecular expression will provide a more focused relation of the diagnosis to the contribution of individual genes or proteins.

Expression of features (for example, the type, size and density of cellular nuclei, or the shape or volume of other defined spaces or structures, such as of blood vessels, etc.) of structural elements therefore represents the basis of diagnosis, which until this time has always been processed in the human mind during visualization. Pathologist-based visual diagnosis relies upon the capacity of the eye to discriminate features of tissue structures, but is heavily weighted toward the human capacity to group elements into patterns that can be discriminated one from another.

Thus, diagnosis is only a moderately accurate predictor of individual prognosis because it is a pooled indicator. To determine the specific relationship of gene expression patterns to tissue diagnosis in each case, more refined correlates are needed—that is, features of structural elements that are induced by individual genes or sets of genes.

To fully effect this desired type of correlation, tissue feature analysis must be carried out in a “hyperquantitative” fashion as disclosed in the present invention. This is done using a combination of machine vision identification of tissue feature elements along with Cartesian mapping and interface systems that enable any possible relationship between the elements to be measured.

The hyperquantitative approach is distinct from the prior art. For example, recent reports have described the use of gene expression profiling for identification of molecular markers of toxicity. However, this technique alone does not account for tissue structural changes, which have been traditionally used by pathologists to discriminate between the types and severity of toxicological response. In a preferred embodiment, the analysis of the combined hyperquantified feature and gene expression profiles of time-varied, control and treated tissues yields more accurate discrimination of affected subjects than analysis of either data set alone, indicating an increased sensitivity of correlated tissue information.

Prior art to elucidate correlations between tissue structures and gene expressions is based on laser-microdissection (LMD). LMD allows collection of specific cell types from a background of cells in a sample by interactively defining areas in a microscope image, and subsequently using lasers to cut out these areas and collect the cells of interest. This method is a destructive method, based on a subjective selection that does not account for the quantities of the structural composition and relationships of structural elements within tissues. LMD also limits the detection of structural elements and discrimination of feature values because of preparation and staining constraints required for subsequent RNA analysis.

The prior art relating to tissue feature analysis is generally limited to the analysis of a single feature or very limited group of features for a structural element. This is distinct from prior applications of image analysis techniques to digital images of stained tissue specimens that have been limited to measures of single elements by single features. We disclose image analysis techniques that may provide objective information on a plurality of tissue features for a plurality of structural elements.

### BRIEF DESCRIPTION OF THE DRAWINGS

FIG. 1. Coordination of Tissue Specimens for Data Collection and Correlation Models FIG. 2a. Principal Component Analysis of gene expression data. FIG. 2b. Principal Component Analysis of feature expression data. FIG. 2c. Principal Component Analysis of combined gene expression and feature set.

FIG. 3. Minkowski distance of gene expression values and image features between subjects in the control, treated, and control vs. treated groups.

### DEFINITION OF CERTAIN KEY TERMS

AOI—Area of Interest. The sub-image where processing/analysis is taking place.

Binary Image—An image comprised of pixels containing two values; one value indicates pixel is in “on” state and the other value indicates pixel is in “off” state.

Colorspace conversion—The process of converting from one color coordinate system to another. Typically, images are represented by three 8-bit planes of information which correspond to the frequency of light sampled to obtain the information; Red, Green, and Blue (RGB). Other colorspace systems are: Lab, YUV, YIQ.

Feature—Mathematical representation quantifying the geometric or biological characteristics of an Object of Interest; e.g., the type, size, texture, shape or density (relative or absolute) of the Object, or the relative presence of a gene or protein within the Object.

Feature Vector—Storage for multiple features extracted from an Object.

Greyscale Image—Image comprised of pixels with multiple intensity values. The intensity value of the pixel is proportional to the amount of photons captured by a photosensitive element.

Hyperquantification—The computer based measurement of one or more structural elements of a tissue to enable a statistical evaluation of: the variability of said measurements according to the classification of the tissue (which may be by its pathology or by the set of subjects, grouped by genotype or phenotype, from which the tissue was obtained); or the causal relationship between those measures and anteceding or concurrent biomolecular activity. The measurements of one or more of these structural elements for any of these purposes is performed by measuring one or more features of the element through the steps of: digital image capture obtained at one or different resolutions, preprocessing through colorspace conversion; segmentation of the processed image to identify Objects of Interest; extraction of feature data expressing the Objects of Interest; and export of extracted feature data to a database. In one embodiment of hyperquantification of cell feature expression, one measures quantitatively the cells of the tissue by at least two features consisting of type and size. In another embodiment of hyperquantification of feature expression of structural elements of liver tissue, one measures quantitatively the hepatocyte nuclei, other nuclei (Kupffer cell nuclei, Ito cell nuclei, inflammatory cell nuclei), sinusoids, vacuoles, and necrotic and inflammatory areas, by the number, area (both absolute and relative), and density of each element.

Image—A 2D array of elements (pixels) representing a scene recorded by a photosensitive device.

OOI—Object of Interest. An Object of Interest may be any structural element of a tissue; e.g., cell, vessel, vacuole, matrix, or boundary layer.

ROI—Region of Interest.

Noncellular space—a vacuole, or other clear space within a tissue having a defined region but not containing any cellular material.

Segmentation—The partitioning of an image into logical groupings; for example, distinguishing nuclei from other biological structures within an image is referred to as nuclei segmentation.

Subject—any living thing; may be human, animal or plant in origin.

Structural Element—a biological component of a tissue; e.g., cell, cell nucleus, cell cluster, necrosis, inflammatory area, matrix, fiber, boundary layer, vessel or noncellular space

Thresholding—Process for converting from a greyscale to a binary image.

Tissue—A group or collection of cells and their intercellular substance.

### SUMMARY OF THE INVENTION

The present invention generally relates to the correlation of hyperquantified tissue feature data expressive of structural elements (“feature expression”) derived from digital images of a set of histopathological specimens obtained from tissues of subjects having shared characteristics with information about molecular activity (which may represent either gene or protein expression) derived from related specimens of the same tissues to provide for the objective characterization and analysis of the variability of tissue features in relation to such molecular activity (the product of such correlation referred to herein as the “correlated tissue information”).

In its broadest embodiment, the present invention is a method of correlating the relative variability of feature expression and gene expression within a set of specimens for subjects with shared characteristics and obtained from tissues of the same type, comprising the steps of quantifying, for at least one structural element of the specimens from each subject, at least one feature selected from a group of features comprising the geometric or biological properties of the structural element; quantifying the gene or protein expression levels obtained within the specimens from each subject; and performing a correlation analysis to determine the relative variability of feature and gene or protein expression within the set of tissue specimens. In a preferred embodiment, correlated tissue information is obtained by creating data sets of tissue feature expression and gene/protein expression that represent more than one time point of treatment or disease and storing those data sets in a database.

By mathematically linking molecular activity and clinical diagnosis through the hyperquantification of tissue feature expression, correlated tissue information provides a more focused and reliable means of identifying potential therapeutic targets and markers and establishing the effect of toxic agents. Such correlated tissue information also allows for comparison and combination with correlated tissue information obtained for other sets of subjects having shared characteristics in common with or dissimilar to the original set, through studies taking place at different times that may have different protocols for the collection, preservation, histological staining or molecular activity assays of the specimens from the other subject sets. The present invention specifically relates to a system and method to obtain and store correlated tissue information by combining hyperquantified tissue feature expression with gene and/or protein expression data. Such a system also allows for the relation of such correlated tissue information to certain clinical diagnoses or conditions through the inclusion or exclusion parameters describing the shared characteristics of the set of subjects providing the tissue specimens for analysis.

Hyperquantification of tissue feature data is a prerequisite to enable the correlation of the composition and organization with the molecular activity of a tissue. The utility of that correlation follows from collecting and storing that data and the related biomolecular expression data by individual subject and tissue. Thus, in a preferred embodiment, the collection of data proceeds from: grouping subjects with shared characteristics; obtaining specimens of tissues of those subjects at more than one time point; and analyzing the specimens to acquire hyperquantified feature data and biomolecular expression data for each tissue at more than one time point. In one embodiment, the specimens may be divided to enable the acquisition of concurrent hyperquantified feature data and biomolecular expression data for each tissue sampled at each time point; however, the schedule may be varied to provide for alternating acquisition of feature data and biomolecular data, provided each tissue is sampled at more than one time point for each data type.

Specifically, the present invention pertains to the correlation of hyperquantified tissue feature data derived from a set of histopathological tissue specimens from subjects with shared characteristics with related gene or protein expression information derived from the same specimen set. The acquisition of the hyperquantified tissue feature data utilizes an improvement of methods of analysis of digital images of histopathological tissue specimens through the improved detection and interpretation of stains in such specimens. The gene or protein expression information is derived from the application of standard microarray assay techniques to related specimens belonging to the same specimen set. The development of an analysis system to robustly identify and quantify features closely characterizing the tissue structural elements allows for closer correlation between the presentations of such elements and concurrent patterns of gene or protein expression.

### DETAILED DESCRIPTION OF THE INVENTION

The assumption is that gene expression and feature expression of structural elements from a single tissue will correlate well but that within a diagnostic category (and across multiple tissue specimens comprising a diagnostic set) there will be substantial variation. Careful interpretation of this variation may lead to disclosure of the specific relationship between a gene (or closely related set of genes) and the tissue element or elements under its (or their) control.

As disclosed herein, hyperquantification is a computer based image interpretation technique that maximizes the biologically relevant feature data that characterizes the structural elements of a tissue. This hyperquantification is done through the steps of: digital image capture obtained at one or different resolutions, preprocessing through colorspace conversion; segmentation of the processed image to identify Objects of Interest; extraction of feature data expressing the Objects of Interest; and export of extracted feature data to a database.

Table 1 shows representative tissue sample data from a group of subjects with shared characteristics. It illustrates theoretical data collected from tissue analysis and genetic analysis of a set of tissues, A-E, for a group of subjects having the same diagnosis. Note that a set of genes and a set of tissue structural feature variables are depicted as having a quantified expression that caries. The analysis illustrates how either ranking of the tissues by relative expression of genetic and tissue features or slope analysis can be used to identify correlates. There are potentially several additional pathways to the identification of correlates, as well.

For purposes of the present invention, shared characteristics can be, for example, same age, same gender, same genotype, same phenotype, same diagnostic criteria established by a particular disease (e.g., cancer disease, obesity, alzheimer's disease, diabetes etc) and such other shared characteristics.

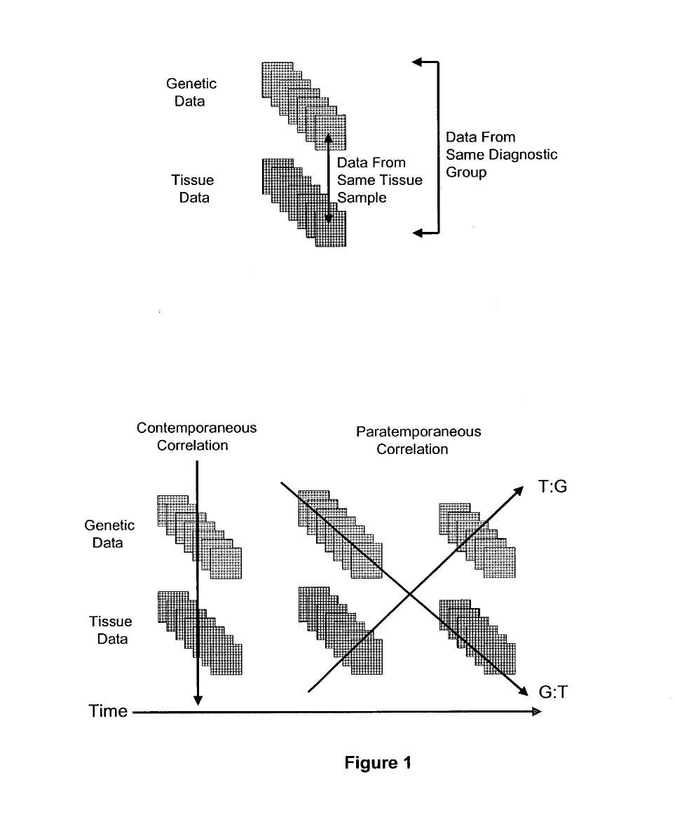

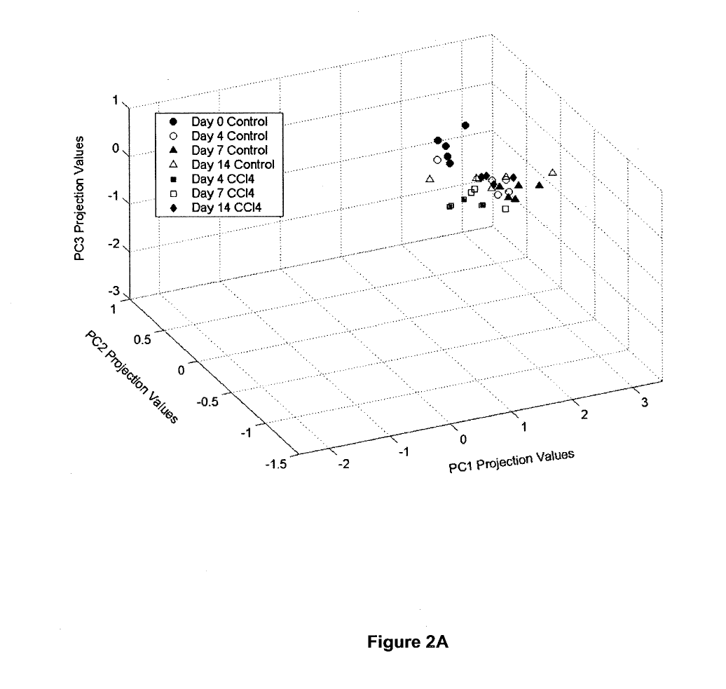

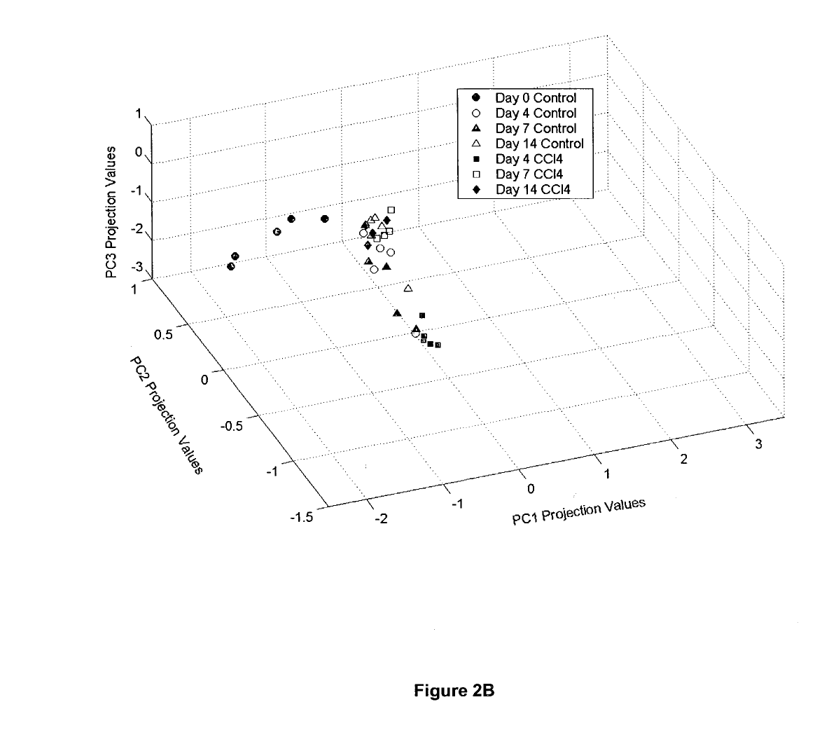

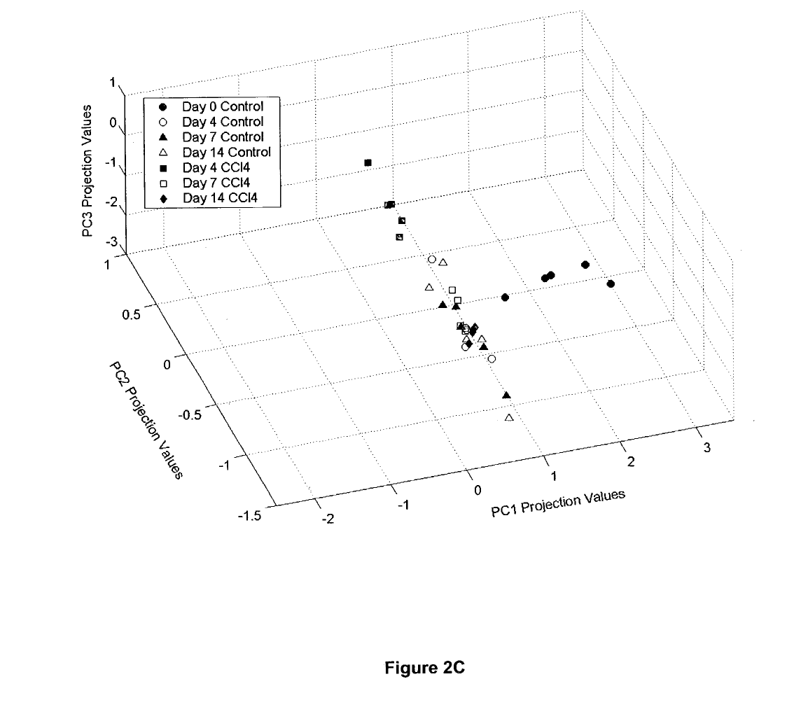

FIG. 1 illustrates how datasets derived from the same tissues within diagnostic groups are organized. In addition, there is the potential that correlation exists between gene expression and tissue structural expression at the same time that the tissue sample is obtained—so called “contemporaneous” correlation. There is also the potential that genes that were turned on in the tissue before the time of sampling correlate more strongly with tissue feature expression because there is a delay in full processing of the genetic expression's impact on the tissue. This could be determined in a time series of tissue sampling, in which genetic expression data from a previous time point is correlated with tissue expression indices at a later time point—so called “paratemporaneous” correlation. A “reverse paratemporaneous” correlation may also occur if tissue features, such as matrix density, influence gene expression in a feedback fashion.

Hyperquantified feature expression data from same time sampling and timed series sampling optimizes the identification of gene correlates to tissue structure. It is very likely that there will be a combination of contemporaneous and the two types of paratemporaneous correlation for any specific tissue specimen set since some genes may control tissue expression over shorter time scales than others. Accurate interpretation of such correlations will require precise description what may be very subtle variability in feature expression of structural elements across the specimen set.

It is important to note that in a ranking system utilizing hyperquantified feature data, the way is also paved for the identification of inverse correlations of gene expression with tissue structural expression. This could occur if a gene were actually an inhibitor of tissue structural expression.

In a preferred embodiment, hyperquantitative tissue analysis is a maximization methodology, since it enables the identification and classification of multiple feature descriptors of tissue structure (the tissue “features”) and their relationships, in order to increase the ability to find correlations with other biological data sets derived from the same tissue. Hyperquantified feature information, derived from time-series data sets, allows application of linear, non-linear, dynamical, synchronous and asynchronous statistical correlation and mathematical modeling of the underlying components and relationships.

A particular element of the present invention is that it correlates data sets representing individual tissues of individual subjects within the reference set. The common use of pooled RNA for gene expression experiments in the prior art clearly compromises the relation of variations in gene expression to particular elements of tissue composition or organization by using data that presents a single score set for multiple specimens. The same problem is presented when the tissue feature data is “pooled” through pathological interpretation that classifies on the basis of overall impression, rather than quantification of the discrete elements.

As one example, a linear correlation of particular tissue feature changes with changes in gene expression would reveal all those genes that show a consistent relationship with these tissue feature changes, and provide a real basis for the identification of drug targets or toxic markers by precisely relating variability in molecular activity with diagnostic cues. Maximization of the hyperquantification of the features of multiple structural elements increases the feedback, “hits” or co-variance that such correlation brings along. Although, in one embodiment, basic correlative conclusions can be drawn when the data is sampled at one control or normal state and one time point of treatment or disease progression, the sensitivity of such analysis is increased if the data is sampled in a time-resolved way that captures the progression and/or regression of tissue structure changes. Other factors that improve the analysis of such correlations include the increase of the spatial resolution to capture these changes and the exclusion of tissue feature or biomolecular expression data that had been averaged or pooled.

An embodiment of the present invention is a method of correlating the relative variability of feature expression and gene expression within a set of specimens for subjects with shared characteristics and obtained from tissues of the same type, comprising the steps of quantifying, for at least one structural element of the specimens from each subject, at least one feature selected from a group of features comprising the geometric or biological properties of the structural element; quantifying the gene or protein expression levels obtained within the specimens from each subject; and performing a correlation analysis to determine the relative variability of feature and gene or protein expression within the set of tissue specimens. In a further embodiment of the present invention utilizing specimens of skin tissue, a number of structural elements, such as cells and boundary layers, are characterized by measurement of multiple tissue features, such as the type, number, area, and density of cells, and the thickness of boundary layers and distribution of cells in relation to such layers, within both normal and pathological tissues. In a further embodiment utilizing specimens of liver tissue, these structural elements include, but are not limited to, hepatocyte nuclei, other nuclei (Kupffer cell nuclei, Ito cell nuclei, inflammatory cell nuclei), sinusoids, vacuoles, and necrotic areas, all of which are characterized by multiple feature measurements, such as the type, number, area (both absolute and relative), and density of cells or necrotic or inflammatory areas.

An embodiment of the present invention is a method to improve the interpretation of tissue responses to potential toxicologic agents by correlating the relative variability of feature expression and gene or protein expression within a set of specimens obtained from tissues of subjects with shared characteristics, where the subjects are grouped in control and test pools, where the subjects grouped in the test pools are further grouped by dosing variations, and where the specimens are obtained at more than one time point. In this embodiment, the method comprises the steps of quantifying, for at least one structural element of the specimens from each subject, at least one feature selected from a group of features comprising the geometric or biological properties of the structural element; quantifying the gene or protein expression levels obtained within the specimens from each subject; and performing a correlation analysis to determine the relative variability of feature and gene or protein expression within the set of tissue specimens for each subject group, and that relative variability is further compared across the results obtained for the each subject group.

Other embodiments may be seen for tissue modeling or in-silico biology. Tissue modeling uses information about structure and function to build computer models which mimic the processes and dynamics in tissues like the formation of layers, mitosis and apoptosis and recovery. Such kind of modeling typically takes into account the structural hierarchies and their interaction. Hyperquantitated feature data correlated with biomolecular expression provides such information to build up structural/functional models.

Outline Of Hyperquantification Approach

Generally, the present invention discloses a novel approach to an automated measurement and analysis system to quantitatively evaluate tissues while reducing the uncertainty, imprecision, time, and subjectivity involved in making manual measurements.

Starting with a color image of a tissue specimen, there are four basic steps in the hyperquantification approach:

1. Colorspace conversion

The first step is to perform a colorspace conversion to provide greater contrast between the Objects of Interest and the rest of the image. One example of a colorspace conversion is a conversion through principal component analysis from a colorspace comprised of red, green, and blue (RGB) intensities into a colorspace representing the predominant colors of the color image.

In one embodiment, the colors of an image might be predominantly created by stains. An example of this might be the blue color imparted by hemotoxylin to enhance observation of cell nuclei. The more traditional method of accomplishing analysis is to make an assumption that the nuclei are always going to be blue. A fixed colorspace transform is selected based on this assumption and applied to the original RGB image to increase the contrast between the blue stain and the rest of the tissue.

The present invention utilizes a principal component based conversion (the Stain Space conversion) to address the weaknesses inherent with using fixed colorspace transforms. The method of the present invention has built in adaptability, meaning, as long as the physical conditions exist (lighting, stain, etc.) to differentiate between the nuclei and other structures, the Stain Space conversion is able to account for shifts of these parameters and produce images that provide contrast between the stained structures (for example, nuclei) and remaining tissue.

2. Segmentation: Identification of Objects of Interest.

The second step is to use the colorspace transformed image and segment the image into 2 parts; target Objects for further analysis and other Objects. Segmenting is effected through conversion of a greyscale image (typically in the range of 0 to 255) to a binary image (effectively, zero or one) by means of thresholding. The result of segmentation is a separate binary image with locations coincident with the pixels associated with the objects of interest being set to the “on” state. The binary image is obtained through thresholding, wherein the pixels contained in the greyscale image are compared against a desired value (the threshold). If the comparison value satisfies a given criterion, the location in the binary image is set to the “on” state; otherwise, the location is set to the off state. The present invention employs an adaptive method for threshold selection based on Bayesian statistics.

3. Extraction of Feature Expression of the Objects of Interest.

The third step of the method is to extract feature expression data for the Objects of Interest. The analysis phase begins with the image being labeled. This process creates uniquely identified Objects (termed blobs by Image Analysis Specialists) about which to extract feature data in order to perform downstream correlation with other forms of information.

The types of features extracted in one embodiment are shape, statistical, and texture. The shape features are derived from the labeled masks (area, roundness, compactness, etc). The statistical features (average pixel intensity for an object, etc.) are derived from the greyscale images considering only pixels co-localized with the locations of the “on” pixels present in the blob image. Texture measures (Edge strength, pixel energy, etc) are calculated after applying digital filters to highlight structures of interest. The extracted features are stored in feature vectors. One feature vector contains the information for one object in the image.

### EMBODIMENTS OF HYPERQUANTIFICATION APPROACH

One embodiment of the present invention is a method by which the location and quantification of features of interest, such as structural features, may be automated wherein the method comprises a step of utilizing a set of stained tissue specimens from multiple donors and the pathological classification of specific features visible within the specimens, such that certain regions in the image are known to represent specific features, such as hepatocyte nuclei or necrotic tissue. One embodiment of the present invention is a method of image processing which includes steps selected from: capturing a color image of a tissue specimen; gathering a background image; calculating a mean value in each band of a background image; using information from the background image to adjust the color image; randomly sampling a fixed number of points from the color image; performing a principal component analysis to obtain three vectors; transforming the color image to a colorspace in which the colors of the colorspace show the type and amount of stain present; and followed by sub-differentiation comprising a step of applying feature values.

The application of feature values may be in cascading sequence. An example of a feature value might be a certain value for cell area, eccentricity or circularity, which value might be selected through experimentation to optimize observation or detection of certain structural elements. One embodiment of the present invention is a method comprising a step of applying a plurality of feature measurement values, which feature values are selected through experimentation to optimize observation of a structural element, wherein the feature values are applied sequentially to make objective determinations to filter out areas of the tissue specimen unlikely to contain the structural element of interest. One embodiment of the present invention is a method of image analysis comprising the steps of quantifying structural elements and differentiating tissue morphologies. Steps of the different embodiments may be combined to form other embodiments.

This ability to clearly define the regions in colorspace that represent the color of a particular stain also allows measurement of the amount of stain at any point in the image, a task which is difficult by other methods. The amount of stain present at any point in the tissue can be determined by calculating the distance in colorspace along the stain curve from the triplet representing the light source to the triplet representing the point in question. The amount of stain can then be calculated. Estimates of biological parameters can then be inferred from the amount of stain present. The most important biological parameters that benefit from this analysis are obviously the features the stain is meant to enhance and for which it is applied. Thus, for example, the ability to accurately detect hematoxylin will improve the ability to detect nuclei in hematoxylin and eosin stained tissue sections.

Examples Of Programs For Generation Of Hyperquantified Feature Data

Multiple methods for generating hyperquantified feature data may be employed to provide for a data set for correlation with gene expression data. Two methods are disclosed here; both methods use the example of liver tissue stained with hematoxylin and eosin.

### EXAMPLE 1

One method for developing automated feature identification and quantification may involve a cascading sequence of segmentation. Within a given tissue-containing image, first necrotic areas are segmented. Through experimentation, the saturation band of the original color image is selected, using standard image analysis tools. The histogram of the saturation band is calculated via a presentation of distribution of pixels according to the particular differentiation criteria, and the total number of pixels from 0 to the mean of the histogram minus the standard deviation of the histogram divided by two is calculated (to define the area of necrosis appearing in the tissue to exclude the area from further analysis). The number of pixels calculated is divided by the total number of pixels in the image, and a percent of “possible necrosis” is found. If this percentage is larger than a prescribed parameter (that, through experimentation, demonstrates that the number of pixels calculated will include all of the area interpreted to represent necrotic tissue in the image), the following necrotic area segmentation is performed. The image is thresholded from 0 to the mean minus the standard deviation of the saturation histogram. The resulting objects are binarized (i.e., converted to a black/white scale), filled (i.e., shapes with incomplete boundaries are enclosed), and a close operation is performed. The boundaries of the resulting objects are extracted (i.e., measured) and those objects larger than a prescribed cutoff parameter are classified as necrotic areas.

The areas identified as necrotic areas are excluded from further segmentation eliminating the possibility of including structural elements within necrotic areas among features identified and quantified thereafter. An embodiment of the invention is a method comprising a step of excluding selected areas of a tissue specimen from further analysis or segmentation. A specific embodiment of the invention is a method comprising a step of excluding necrotic area from consideration for analysis of certain structural elements.

Red cells and blood-filled objects are next segmented by thresholding the original color image from 0 to the mean plus the standard deviation of the histogram in the red band (a measure of the distribution of pixels of the image within the band) from 0 to the mean minus the standard deviation of the histogram in the green band (a measure of the distribution of pixels of the image within the band), and from one half of the mean to the mean minus the standard deviation of the histogram in the blue band (a measure of the distribution of pixels of the image within the band). All resulting objects are classified as red cell objects.

Next, all serum-filled and white colored objects are segmented. Through experimentation, this segmentation was accomplished by thresholding from the mean to the mean plus the standard deviation of the histogram in the green band. All resulting objects are classified as serum objects.

Next, dark objects are segmented from the red band by thresholding from 0 to the mean minus the standard deviation of the histogram in the red band.

Once certain groups of objects are identified through image segmentation, further differentiation within those groups is achieved by applying morphological parameters. These parameters are optimized experimentally. If red cell objects are found, single red blood cells are classified as red cell objects with sizes between two specified parameters. Similarly, if red cell objects are found, large blood vessels are classified as red cell objects larger than a specified parameter. Also, if serum objects are found, those serum objects larger than the same size parameter are classified as large blood vessels, as large blood vessels can be filled with serum, blood, or empty in histological sections.

All large blood vessels are recreated and dilated a number of times specified by the user. The resulting area is classified as the boundary of all large blood vessels. This enables error to tend toward finding more large blood vessels, rather than missing some (meaning that the method is oriented to select false positives).

If red cell objects, serum objects, and dark objects are found, the three areas are recreated and filled. A close operation is performed (to complete the boundaries of all identified objects), and the resulting areas that are larger than a prescribed parameter are classified as portal tracts. If portal tracts are found, these objects are recreated and dilated a number of times specified by the user. The resulting area is classified as the boundary of all portal tracts. This enables error to tend toward finding more portal tracts, rather than missing some (meaning the method is oriented to select false positives).

If dark objects are found, these are recreated and filled. If any of these has an area larger than a prescribed parameter, it is labeled as a tissue fold (meaning a fold of the tissue specimen). All tissue folds are recreated alone and dilated a number of times prescribed by the user.

All necrotic areas, large blood vessel areas, red blood cell areas, portal tract areas, and tissue fold areas are excluded from further segmentation and classification.

For both hepatocyte nuclei and other nuclei, the red band is selected as the ideal band for segmentation, and segmentation is performed by classifying all pixels with values between 0 and the mean minus the standard deviation of the histogram in the red band. If selected by the user, double nuclei (peanut shapes) (meaning something shaped like a peanut) are separated using pre-determined size and eccentricity thresholds (the size threshold is set to a predetermined area; the eccentricity threshold measures the deviation of the shape from a perfect circle). Also, if selected by the user, a convex hull can be performed to ensure that only convex shaped nuclei are identified. Nucleus classification is performed by plotting the standard deviation of the intensity values within each nuclei object versus its respective size, and separating the plot into two groups either manually or automatically by maximizing the cluster distance between the two groups and generating a line between their centroids.

Serum areas are segmented from the green band by thresholding from the mean plus the standard deviation of the histogram in the green band to a particular number, such as 255. (in one standard for color segmentation, 0 is black and 255 is white). These serum objects are classified into either serum-filled sinusoids or vacuoles. The classification of the serum objects is performed morphologically by classifying vacuoles as serum objects with circularity values less than a prescribed parameter. Thus, an embodiment of the present method may include a step in which image analysis establishes morphological differentiation. Serum-filled sinusoids and blood-filled sinusoids are combined to form the set of identified sinusoids. The parameters which have been selected for this embodiment are given in Table 2.

### EXAMPLE 2

1. Stain Space Image Calculation

a. Get 24-bit tissue image (Liver) and associated background image (captured from the microscope with no tissue present)

b. Sample the liver image for N number of pixels

a. Where N should be large enough to represent the image statistically

b. Perform a chromatic density filter on the image, rank-order the resulting density magnitudes and choosing sample points from the lower quartile and upper quartile of the ranked values

c. Choosing only those sample points that exceed a distance calculation for chromatic separation from the background image mean value

c. Calculate the representative 24-bit stain space image, where each plane of the stain image corresponds to the optical stain absorption property for each stain component (hematoxylin, eosin)

2. Nuclei Segmentation

a. Get 24-bit tissue image (Liver) and associated background image (captured from the microscope with no tissue present)

b. Perform Stain Space Image Calculation

c. Select the Hematoxylin image representation from the Stain Image, let this image be represented by symbol Iₒ

d. Perform an order 0 Rank Filter on Iₒ to make dark objects prominent

e. Apply a series of convolution kernels to image Iₒ to further isolate potential objects in the image representative of Nuclei and remove associated noise

f. For each isolated object Oₒ perform a classification to determine if the object is Hepatocellular nuclei or other dense basophilic object

i. Gaussian filter Iₒ to reduce the effects of noise

ii. Compute moment statistics of gradient magnitude change summed over the region covered by object Oₒ

iii. Compute moment statistics of gradient direction change summed over the region covered by object O₀

iv. Utilize Bayesian statistical methods to determine the Probability that object Oₒ belongs to class Hepatocyte using a likelihood function derived from prior training set data

h. Whitespace Segmentation (Vacuole and Sinusoidal Area)

i. Generate image I₁ by inverting the summed Hematoxylin and Eosin image bands

ii. Convolve I₁ with various kernels to determine edge magnitude

iii. Determine regions of low magnitude change via density measurement and generate a histogram of the associated pixel intensity values for all regions

iv. Threshold image I₁ by utilizing the mean value minus one standard deviation, taking all pixel values greater to represent whitespace, generating image I₂

v. Perform morphology analysis on image I₂ selecting features of area and compactness to denote Vacuole or Sinusoidal classification

1. Area>=Areaₘᵢₙ and 1<=Compactness <=1.8 are classified as Vacuoles

2. Others are classified as Sinusoids

Example Of Generation And Use Of Hyperquantifled Feature Data For Generating Correlated Tissue Information

The following is an example of the use of hyperquantified feature data for gene expression correlation in a toxicological study. The example demonstrates that the use of hyperquantified data in conjunction with gene expression data of the same tissues, employing different statistical techniques, improves the classification of subjects, improves the detection of outliers, and establishes relationships between particular genes and tissue features.

I. Establishment of Diagnostic Set of Tissue Specimens. Experimental Design.

A diagnostic tissue specimen set was composed from seven groups of male Sprague-Dawley rats, 6 weeks of age and weighing between 200 and 225 g at the onset of the study (n=5 per group). The first group of five rats was euthanized after 12 hours of overnight fasting. The other six groups were separated into three groups of vehicle controls (days 4, 7, and 14), and three groups of CC14-treated animals (days 4, 7, and 14). Each rat from these six groups was administered, using intraperitoneal injection once a day, either pure corn oil (vehicle control animals) or CC14 dissolved in corn oil (˜15% v/v) at a dose level of 1000 mg/kg/day, a level that has been shown to be hepatotoxic but not lethal. Animals in the day 4 groups were dosed for three consecutive days, subsequently fasted for one day, and euthanized after day 4. Animals in the day 7 groups were dosed for three consecutive days, allowed to feed normally for three consecutive days, fasted for one day, and euthanized after day 7. Finally, animals in the day 14 groups were dosed for three days, allowed to feed normally for ten days, fasted for one day, and euthanized after day 14.

II. Development of Hyperquantified Feature Data

The central lobe of each liver was harvested at time of necropsy, and approximately 1 g was weighed, and flash frozen by immersion in liquid nitrogen for subsequent RNA extraction. The remaining fraction of the liver was placed in 10% formalin. A single representative section was cut from each block, placed on a slide, and stained with H&E. Digital images of each slide were acquired using a research microscope and digital camera (Olympus E600 microscope and Sony DKC-ST5). These images were acquired at 20×magnification with a resolution of 0.64 mm/pixel. Digital histopathology was performed on the resulting images.

First a digital image analysis identified and annotated Objects of Interest in a tissue using machine vision. These Objects, that are structural elements of the tissue, can be annotated because they are visually identifiable and have a biological meaning like hepatocytes, sinusoids, vacuoles. Subsequently a quantification of these structures by features like area or stain intensities and their relationship to the field of view or per unit area in terms of a % coverage was performed. The parameters resulting from this hyperquantification that were used is this example are given in Table 2 [Ranking of PCA values of gene expression and tissue feature expression]. Features for hyperquantification will be specific for each structural element of each tissue type, and may also include relations between features, measures of overall heterogeneity, including orientation, relative locations, and textures. Additional feature information for a tissue increasing the robustness of the hyperquantification, may be gained by using different imaging techniques, including phase contrast, darkfield, multispectral, and fluorescence, with or without specific staining techniques that can enable or improve the detection features over hematoxylin and eosin staining and brightfield microscopy.

III. Development of Gene Expression Data

As two biochips were run for each animal, and three replicates of each oligonucleotide existed on each biochip, the resulting data set consisted of six data points for each gene or control probe. The “raw” data of gene expression values, gene names, and biochip names (animal names) from each biochip (2 per animal) was imported into Matlab Rel. 12.1. An outlier detection filter was applied to the data. First, the median of each set of 6 data points was calculated, then the absolute value of the difference between each data point and the median of the set to which it belonged was calculated. The median absolute deviation was then calculated. A modified z-score (Mi) for each original gene expression value was derived as the absolute difference between the original data point and the median of the set to which it belonged multiplied by 0.6745 and divided by the median absolute difference for the set. Then, if a gene expression value in a given set of 6 values was the largest of the values (outliers tend to exhibit larger than normal intensity values due to dust on the biochip) and its Mi was larger than D=8.7 (<0.5% of all data points exhibit significant outlier character), it was labeled an outlier and removed from the raw data set. The median of the remaining gene expression values for each probe on the biochip was then calculated and used for further analysis. This resulted in one gene expression value per gene per animal.

IV. Data Correlation and Results

In order to correlate tissue feature and gene expression data, the following normnalization was performed: Gene expression values were normalized for the maximum value of gene expression across all genes. Tissue features were normalized on a per feature basis. The latter was necessary since the nature of the tissue features is quite different, such as an absolute number of pixels, % value, variation etc.

Based on these normalized values, a Principal Component Analysis (PCA) was performed using Matlab. The first three eigenvalues of each animal were calculated and displayed. FIGS. 2a, 2b, and 2c show three PCA plots: FIG. 2a shows the gene data only, FIG. 2b the image data, and FIG. 2c the combined set. Since all three figures use the same scaling, these PCA plots can be directly compared. It is obvious that the combined set shows the best differentiation, in particular the control group on day 0 and the day 4 treated groups are better separated in the combined set than they are on the gene expression or tissue metrics alone.

Correlation Between Tissue Features and Gene Expression Values

As an example, principal component analysis was performed on the gene expression values after outlier removal, normalization, and removal of control probes. The coefficients corresponding to the relative contribution of each gene to each of the first three principal components were exported. The sum of the absolute values of these three coefficients for each gene was calculated. The genes were then sorted in descending order suggesting a rank order of importance to the patterns observed in the three-dimensional PCA plot. The same procedure was carried out for the normalized tissue feature data set. These two lists, the gene list and the tissue feature list, were subsequently combined (see Table 2). This method detected those genes and features which are significant and distinct from all other tissue features and gene expression values, and they are the characteristic markers of the variability in the tissue. A similarity of genes and tissue features having high ranks suggests that they are correlated. Animals of all time points, control and treated, contributed to this analysis.

Table 3 presents a different correlation based on Spearman's rank analysis. For the application of this method a normalization of gene expression and tissue features is not required. This rank correlation can be performed at different levels of confidence (t-levels) that allow the researcher to draw conclusions about the degree of correlation, and also allows the researcher to cut down the list of genes to a reasonable number to facilitate further investigations.

Hyperquantitative tissue analysis, representing a rich set of structural features of the tissue, is an important prerequisite to run this type of correlation, because it is not known prior to the analysis, which of the features are markers of the treatment or disease.

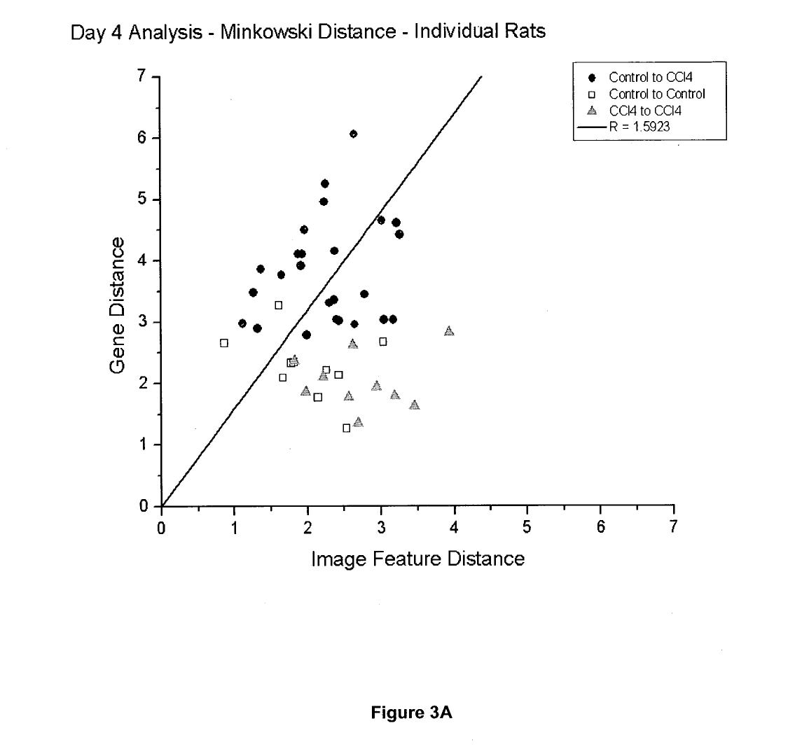

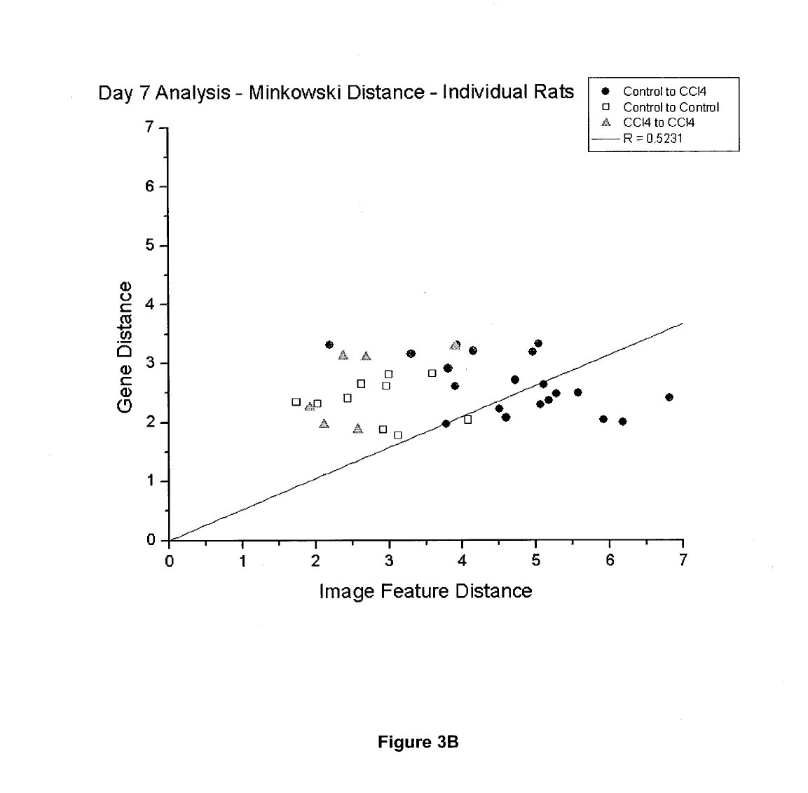

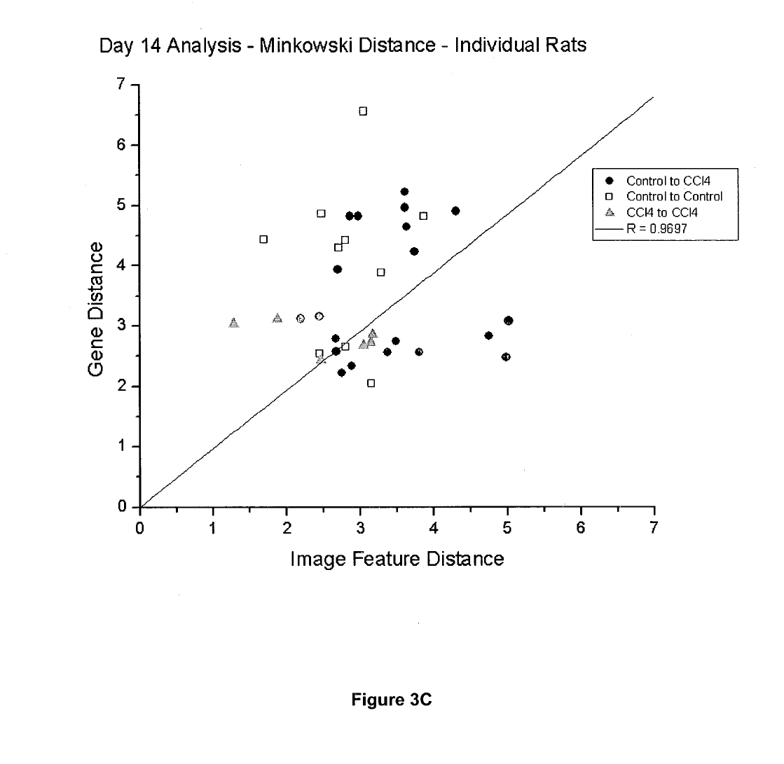

The relation between gene expression and hyperquantitative tissue feature data may be linear or non-linear, in synchronous or asynchronous arrangements. One way of detecting asynchronies is demonstrated in FIG. 3, This figure shows Minkowski distances of gene expression values and feature expression values between animals in the control, treated and control vs. treated groups: FIGS. 3A, B and C show data from animals sacrificed at day 4, day 7 and day 14, respectively. Asynchronies in relations of gene expression to tissue feature expression are revealed and outliers are detected.

For the determination of Minkowski distances, the difference of all parameters (such as all gene expression values) between two animals, such a control and a treated animal, are determined. After normalization of the data as described above, Minkowski distances for the gene data were plotted against the Minkowski distance of the hyper-quantitative tissue features on a animal-to-animal basis. Straight lines on each plot indicate the regression of the combined set of data. Comparing FIG. 2a through 2c demonstrates the difference in impact of tissue profiles for genes and tissue features. Gene features are driving the scatterplot on day 3, but the image features are dominant on day 7, while on day 14 both the gene and tissue features have about the same impact. Animals that do not fall into the main clusters are considered to be outliers, and are of particular interest in toxicological studies. An embodiment of the invention is a method for determining the relative variability of structural elements and gene or protein expression within a selected tissue sample, comprising the steps of quantifying structural elements of the tissue sample to obtain tissue structure data, wherein the number of structural elements is a number selected from the group consisting of three, four, five, six and seven; quantifying levels of the gene expression in the tissue sample to obtain gene or protein expression data (gene expression profiling); normalizing the tissue structure and gene or protein expression datasets; and performing a correlation analysis based on normalized values to determine relative variability of structural elements and gene or protein expression in the tissue sample. An embodiment of this method is wherein the structural elements are determined by obtaining tissue structure data by performing histomorphometric analysis of the tissue sample. An embodiment of this method is wherein the gene expression data of the tissue sample are obtained by gene expression profiling using nucleic acid microarray chips. An embodiment of this method is wherein the gene expression profiling comprises analysis of species of various RNAs or cDNAs. An embodiment of this method is wherein the gene expression profiling comprises analysis of quantities of various RNAs or cDNAs.

Another embodiment of this invention is a method for ascertaining progression or regression of a disease in a given patient population following a treatment for the disease, the method comprising the steps of: obtaining gene expression data and tissue structure data of a selected tissue sample from the selected patient population; obtaining gene expression data and tissue structure data of the corresponding tissue sample from a control population without the disease; normalizing each of said data to obtain normalized values; obtaining tissue structure data by performing histomorphometric analysis of the tissue set; obtaining hyperquantified tissue information based on gene expression data and tissue structure data for the patient population and the control population; and determining the progression or regression of the disease by making comparisons between said tissue information for the patient population and the control population. An embodiment of the method is wherein the disease is selected from the group consisting of cancer, Alzheimer's disease, and PCOS. An embodiment of the method is wherein the tissue is selected from the group consisting of brain tissue, skin tissue, lung tissue, and liver tissue. An embodiment of the method is wherein the tissue structure data is obtained by performing histomorphometric analysis of the tissue sample. An embodiment of the method is wherein the treatment for the disease is a pharmaceutical treatment, physical treatment or a gene therapy treatment.

Another embodiment of the invention is a method for determining drug toxicity in a selected tissue sample comprising the steps of: hyperquantifying tissue information of the tissue sample from individuals exposed to a given drug at a given dose; hyperquantifying tissue information of the corresponding tissue sample from control individuals who are not exposed to said drug; and determining drug toxicity by making comparisons between hyperquantified tissue information from individuals exposed to the drug and the control individuals. An embodiment of this method is wherein the hyperquantification involves the computer based measurement of more than three structural elements. An embodiment of this method is wherein the hyperquantification involves the computer based measurement of more than four structural elements. An embodiment of this method is wherein the hyperquantification involves the computer based measurement of five structural elements. An embodiment of this method is wherein the tissue sample is a liver tissue sample. An embodiment of this method is wherein the tissue structure data is obtained by performing histomorphometric analysis of the tissue sample.

Another embodiment of the invention is a method of correlating the relative variability of structural elements and gene or protein expression within a set of tissue specimens, comprising the steps of quantifying at least three structural elements selected from the group of structural elements comprising cell, cell nucleus, cell cluster, necrotic area, inflammatory area, matrix, fiber, boundary layer, vessel and noncelluar space, obtained from a set of tissue specimens from an entity; quantifying the gene or protein expression levels obtained within the set of tissue specimens from the entity; and performing a correlation analysis to determine the relative variability of structural features and gene or protein expression within the set of tissue specimens. An embodiment of this method is wherein Minkowski distances for the gene expression data are plotted against the Minkowski distances for the quantified structural elements on an entity by entity basis. An embodiment of this method is wherein the structural element information and the gene or protein expression data are measured at more than one time. An embodiment of this method is wherein the structural element information is measured at three times (a, b, c) and the gene or protein information is measured at three times (d, e, f). In one embodiment of this method a=d, b=e, and c=f.

All publications and patent applications mentioned in the specification are indicative of the level of those skilled in the art to which this invention pertains. The contents of all publications, patents and patent applications, including the U.S. patent application Ser. Nos. 09/338,909, 09/338,908, 10/106,582, and 10/158,486 are herein incorporated by reference to the same extent as if each individual publication, patent or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

TABLE 1
Representative Tissue Sample Data From A Group of Subjects With Shared Characteristics
Tissue Feature Expression Data

| | A | B | C | D | E | | For Slope Analysis: | | | | | | |
|---|---|---|---|---|---|---|---|---|---|---|---|---|---|
| | Feature | Feature | Feature | Feature | Feature | Rank (Low | Feature Expression Intensity By | | | | | | |
| | Expression | Expression | Expression | Expression | Expression | To High) | Rank | | | | | | |
| Feature | Intensity | Intensity | Intensity | Intensity | Intensity | Tissue | Feature | 1 | 2 | 3 | 4 | 5 | Slope |
| Z | 4 | 2 | 8 | 12 | 10 | BACED | Z | 2 | 4 | 8 | 10 | 12 | 2.6 |
| Y | 2 | 3 | 1 | 2 | 1 | C/E A/D B | Y | 1 | 1 | 2 | 2 | 3 | 0.5 |
| W | 3 | 15 | 6 | 12 | 9 | ACEDB | W | 3 | 6 | 9 | 12 | 15 | 3 |
| V | 4 | 1 | 3 | 6 | 12 | BCADE | V | 1 | 3 | 4 | 6 | 12 | 2.5 |
| U | 6 | 2 | 3 | 5 | 1 | EBCDA | U | 1 | 2 | 3 | 5 | 6 | 1.3 |

Gene Expression Data

| | Expression | Expression | Expression | Expression | Expression | | | | | | | | |
|---|---|---|---|---|---|---|---|---|---|---|---|---|---|
| Gene | Intensity | Intensity | Intensity | Intensity | Intensity | Genetic | Gene | | | | | | Slope |
| Alpha | 30 | 40 | 16 | 8 | 12 | DECAB | Alpha | 8 | 12 | 16 | 30 | 40 | 8.2 |
| Beta | 10 | 15 | 5 | 10 | 5 | C/E A/D B | Beta | 5 | 5 | 10 | 10 | 15 | 2.5 |
| Gamma | 30 | 150 | 60 | 120 | 90 | ACEDB | Gamma | 30 | 60 | 90 | 120 | 150 | 30 |
| Delta | 8 | 60 | 20 | 2 | 100 | DACBE | Delta | 2 | 8 | 20 | 60 | 100 | 24.8 |
| Epsilon | 120 | 40 | 60 | 100 | 20 | EBCDA | Epsilon | 20 | 40 | 60 | 100 | 120 | 26 |

TABLE 2
Ranking of PCA values of gene expression
and tissue feature expression.

| Tissue Feature | Gene | PCA Value |
|---|---|---|
| Vacuoles/mm\hat{ }2 | | 0.92814153 |
| Area % Vacuoles | | 0.80617922 |
| | RATCYPJ_0_0_926 | 0.6001254 |
| % WS | | 0.59119033 |
| Hep Nuclei Size Variance | | 0.56130343 |
| | RATGLTA_0_0_1 | 0.5062008 |
| Area % Sinusoids | | 0.46427549 |
| Sinusoids/mm\hat{ }2 | | 0.44400308 |
| Vacuole Size Variance | | 0.43621823 |
| | RATMETA_0_0_330 | 0.4243633 |
| Area % Hep Nuclei | | 0.41425827 |
| Area % Total Nuclei | | 0.40815322 |
| | RATCYP45D_0_0_1634 | 0.40345915 |
| Other Nuclei/mm\hat{ }2 | | 0.35499873 |
| Sinusoid Size Variance | | 0.35336786 |
| | RNPAIHC3_0_0_2145 | 0.344661008 |
| | RATCY45AB_3_0_134 | 0.33452892 |
| Hep Nuclei/mm\hat{ }2 | | 0.30484671 |
| Area % Other Nuclei | | 0.29524044 |
| | RNU44845_0_0_1359 | 0.28665032 |
| | RATIVAA_0_0_382 | 0.28520978 |
| | RATSBACT_0_0_1410 | 0.27469077 |
| % Haem | | 0.27150617 |
| | RNRPS29_0_0_147 | 0.26343431 |
| | RATGST_0_0_295 | 0.23393978 |
| | RATLADA_0_0_914 | 0.214069463 |
| | RATCP450_0_0_1100 | 0.212550392 |

TABLE 3
Result of a Spearman's rank correlation. Depending on
the level of significance, different numbers of genes were
found to correlate with particular tissue feature expression.

| | Critical T-Value | |
|---|---|---|
| | 2.056 | 2.779 |
| | 95% confidence interval (t- | Number of |
| | test) Number of Significant | Significant Gene |
| Tissue Metric | Gene Comparisons | Comparisons |
| % WS | 284 | 122 |
| % Haem | 56 | 9 |
| Sinusoids/mm\hat{ }2 | 72 | 14 |
| Vacuoles/mm\hat{ }2 | 194 | 52 |
| Hep Nuclei/mm\hat{ }2 | 60 | 10 |
| Other Nuclei/mm\hat{ }2 | 143 | 45 |
| Total Nuclei/mm\hat{ }2 | 100 | 19 |
| Area % Sinusoids | 191 | 77 |
| Area % Vacuoles | 189 | 53 |
| Area % HepNuclei | 112 | 41 |
| Area % OtherNuclei | 68 | 13 |
| Area % TotalNuclei | 86 | 28 |
| Sinusoid Size Variance | 86 | 16 |
| Vacuole Size Variance | 137 | 38 |
| HepNuclei Size Variance | 122 | 31 |
| Total | 2311 | 568 |

What is claimed is:

1. A method of determining correlation of relative variability of structural elements and gene expression within a set of tissue specimens for subjects having shared characteristics and obtained from tissues of the same type, comprising the steps of
quantifying, for at least one structural element obtained from the set of tissue specimens, at least one feature selected from the group consisting of geometric properties of the structural element and biological properties of the structural element;
quantifying gene expression levels obtained within the set of tissue specimens; and
performing a correlation analysis in order to determine the correlation of the relative variability of the selected said elements as characterized by the selected feature and gene expression within the set of tissue specimens.

2. A method of determining correlation of relative variability of structural elements and gene expression within a set of tissue specimens for subjects having shared characteristics and obtained from tissues of the same type, comprising the steps of
quantifying, for at least one structural element selected from the group consisting of cells, cell nuclei, cell clusters, fibers, inflammatory area, matrix, necrosis, vessels, boundary layers and noncellular spaces obtained from the set of tissue specimens, at least two features selected from the group consisting of type, size, texture, shape and density within the structural element;
quantifying the gene expression levels obtained within the set of tissue specimens; and
performing a correlation analysis in order to determine the correlation of the relative variability of the selected elements as characterized by the selected features and gene expression within the set of tissue specimens.

3. The method of claim 2, wherein the structural element is cells and the features are type and density.

4. The method of claim 2, wherein the structural element is cell nuclei and the features are type and density.

5. The method of claim 2, wherein the structural elements is cells and the features are type, size and density.

6. The method of claim 2, wherein the structural elements is cell nuclei and the features are type, size and density.

7. The method of claim 2, wherein the structural element is cells and the features are type, size, shape and density.

8. The method of claim 2, wherein the structural element is cell nuclei and the features are type, size, shape and density.

9. A method of determining correlation of relative variability of structural elements and gene expression within a set of tissue specimens for subjects having shared characteristics and obtained from tissues of the same type, comprising the steps of
quantifying, for at least two structural elements selected from the group consisting of elements comprising the cells, cell nuclei, cell clusters, fibers, inflammatory area, matrix, necrosis, vessels, boundary layers and noncellular spaces obtained from the set of tissue specimens, at least two features selected from the group consisting of type, size, texture, shape and density (absolute or relative) within the structural element;
quantifying the gene expression levels obtained within the set of tissue specimens; and
performing a correlation analysis in order to determine the correlation of the relative variability of the selected elements as characterized by the selected features and gene expression within the set of tissue specimens.

10. The method of claim 9, wherein the structural elements are cells and cell clusters and the features are type and density.

11. The method of claim 9, wherein the structural elements are cells and vessels and the features are type and density.

12. The method of claim 9, wherein the structural elements are cells and noncellular spaces and the features are type and density.

13. A method of correlating the relative variability of structural elements and protein expression within a set of tissue specimens for subjects having shared characteristics and obtained from tissues of the same type, comprising the steps of
quantifying, for at least one structural element obtained from the set of tissue specimens, at least one feature selected from the group consisting of geometric properties of the structural element and biological properties of the structural element;
quantifying the gene expression levels obtained within the set of tissue specimens; and
performing a correlation analysis in order to determine the correlation of the relative variability of the selected elements as characterized by the selected features and gene expression within the set of tissue specimens.

14. A method of determining correlation of relative variability of structural elements and protein expression within a set of tissue specimens for subjects having shared characteristics and obtained from tissues of the same type, comprising the steps of
quantifying, for at least one structural element selected from the group consisting of elements comprising the cells, cell nuclei, cell clusters, fibers, inflammatory area, matrix, necrosis, vessels, boundary layers and noncellular spaces obtained from the set of tissue specimens, at least one feature selected from the group consisting of features comprising the type, size, texture, shape and density (absolute or relative) within the structural element;
quantifying the gene expression levels obtained within the set of tissue specimens; and
performing a correlation analysis in order to determine the correlation of the relative variability of the selected elements as characterized by the selected feature and gene expression within the set of tissue specimens.

15. The method of claim 14, wherein the structural element is cells and the features are type and density.

16. The method of claim 14, wherein the structural elements is cells and the features are type, size and density.

17. The method of claim 14, wherein the structural element is cells and the features are type, size, shape and density.

18. A method of determining correlation of relative variability of structural elements and protein expression within a set of tissue specimens for subjects having shared characteristics and obtained from tissues of the same type, comprising the steps of
quantifying, for at least two structural elements selected from the group consisting of cells, cell nuclei, cell clusters, fibers, inflammatory area, matrix, necrosis, vessels, boundary layers and noncellular spaces obtained from the set of tissue specimens, at least two features selected from the group consisting of type, size, texture, shape and density (absolute or relative) within the structural element;
quantifying the gene expression levels obtained within the set of tissue specimens; and
performing a correlation analysis in order to determine the correlation of the relative variability of the selected features and gene expression within the set of tissue specimens.

19. The method of claim 18, wherein the structural elements are cells and cell clusters and the features are type and density.

20. The method of claim 18, wherein the structural elements are cells and vessels and the features are type and density.

21. The method of claim 18, wherein the structural elements are cells and noncellular spaces and the features are type and density.

22. A method of determining correlation of relative variability of structural elements and gene expression within a set of tissue specimens for subjects having shared characteristics and obtained from tissues of the same type, comprising the steps of
quantifying, for at least two structural elements selected from the group consisting of cells, cell nuclei, cell clusters, fibers, inflammatory area, matrix, necrosis, vessels, boundary layers and noncellular spaces obtained from the set of tissue specimens, at least two features selected from the group consisting of type, size, texture, shape and density within the structural element;
quantifying the gene expression levels obtained within the set of tissue specimens; and
performing a correlation analysis in order to determine the correlation of the relative variability of the selected elements as characterized by the selected features and gene expression within the set of tissue specimens;
where the specimens are acquired at least at two time points.

23. The method of claim 22, wherein the structural elements are cells and cell clusters and the features are type and density.

24. The method of claim 22, wherein the structural elements are cells and vessels and the features are type and density.

25. The method of claim 22, wherein the structural elements are cells and noncellular spaces and the features are type and density.

26. A method of determining correlation of relative variability of structural elements and gene expression within a set of tissue specimens for at least two groups of subjects having shared characteristics and obtained from tissues of the same type, comprising the steps of
quantifying, for at least two structural elements selected from the group consisting of elements comprising the cells, cell nuclei, cell clusters, fibers, inflammatory area, matrix, necrosis, vessels, boundary layers and noncellular spaces obtained from the set of tissue specimens, at least two features selected from the group consisting of features comprising the type, size, texture, shape and density within the structural element;
quantifying the gene expression levels obtained within the set of tissue specimens; and
performing a correlation analysis in order to determine the correlation of the relative variability of the selected elements as characterized by the selected features and gene expression within the set of tissue specimens;
where the specimens are acquired at least at two time points, t1 and t2 or tn, the subject groups are a control and at least one study group, and the study group has been administered a compound to test its toxicity.

27. The method of claim 26, wherein the structural elements are cells and cell clusters and the features are type and density.

28. The method of claim 26, wherein the structural elements are cells and vessels and the features are type and density.

29. The method of claim 26, wherein the structural elements are cells and noncellular spaces and the features are type and density.

30. The method of claim 26, wherein the tissue type is liver, the structural elements are cells and cell clusters and the features are type and density.

31. The method of claim 26, wherein the tissue type is liver, the structural elements are cells and vessels and the features are type and density.

32. The method of claim 26, wherein the tissue type is liver, the structural elements are cells and noncellular spaces and the features are type and density.

33. The method of claim 26, wherein the specimens acquired at t1, t2 or tn are alternatively used to quantify gene expression data and feature expression data.

34. The method of claim 26, wherein the specimens acquired at t1, t2 or tn are alternatively used to quantify gene expression data and feature expression data.

35. The method of claim 26, wherein the specimens acquired at t1, t2 or tn are alternatively used to quantify gene expression data and feature expression data.
